(19) United States
(12) Patent Application Publication
(10) Pub. No.: US 2016/0178585 A1
(43) Pub. Date: Jun. 23, 2016

(54) DEVICE FOR DETECTING AIR POLLUTANT AND METHOD THEREOF

(71) Applicant: HON HAI PRECISION INDUSTRY CO., LTD., New Taipei (TW)

(72) Inventors: CHENG-CHING CHIEN, New Taipei (TW); JUN-JIN WEI, New Taipei (TW)

(21) Appl. No.: 14/819,693

(22) Filed: Aug. 6, 2015

(30) Foreign Application Priority Data
Dec. 17, 2014 (CN) 201410790355.5

(51) Int. Cl. G01N 33/00

(52) U.S. Cl. CPC G01N 33/0004

(57) ABSTRACT

A method for detecting an air pollutant includes feeding air into a device to detect a quantity of the air pollutant, determining whether the quantity of the air pollutant exceeds a threshold value, turning on a gyroscope and an accelerometer of the device when the quantity of the air pollutant exceeds the threshold value, obtaining positions and moving distances of the device from the gyroscope and the accelerometer, calculating a relationship between the positions and moving distances of the device and the quantity of the air pollutant, and displaying the relationship between the positions and moving distances of the device and the quantity of the air pollutant on a display.

### FIELD

The subject matter herein generally relates to air pollutants, and more particularly to a device for detecting a quantity of an air pollutant and a method thereof.

### BACKGROUND

Electronic devices such as smart phones may include a gyroscope and an accelerometer to detect positions and moving distances of the device. If the device can further include an air pollution detecting function, it can greatly help a user thereof to escape from the source of the air pollution.

### BRIEF DESCRIPTION OF THE DRAWINGS

Implementations of the present technology will now be described, by way of example only, with reference to the attached figures.

FIG. 1 is a block diagram of an embodiment of a device for detecting an air pollutant.

FIG. 2 is a diagrammatic view of the device of FIG. 1.

FIG. 3 is a diagrammatic view of calculating a quantity of the air pollutant relative to a horizontal orientation of the device.

FIG. 4 is a diagrammatic view of calculating a quantity of the air pollutant relative to a vertical orientation of the device.

FIG. 5 is a diagrammatic view of calculating a relationship between the quantity of the air pollutant and positions and moving distances of the device.

FIG. 6 is a flowchart of an embodiment of a method for detecting an air pollutant.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

In general, the word “module” as used hereinafter refers to logic embodied in hardware or firmware, or to a collection of software instructions, written in a programming language such as, for example, Java, C, or assembly. One or more software instructions in the modules may be embedded in firmware such as in an erasable-programmable read-only memory (EPROM). It will be appreciated that the modules may comprise connected logic units, such as gates and flip-flops, and may comprise programmable units, such as programmable gate arrays or processors. The modules described herein may be implemented as either software and/or hardware modules and may be stored in any type of computer-readable medium or other computer storage device.

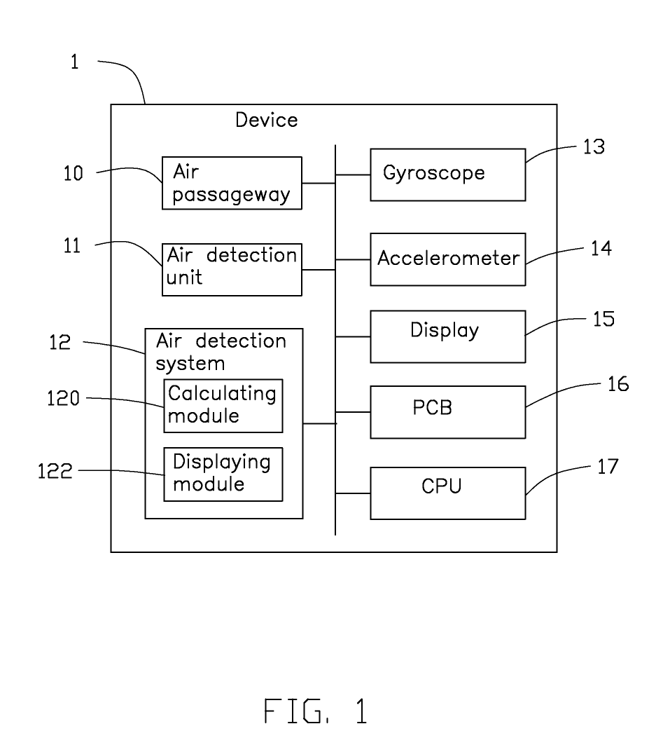

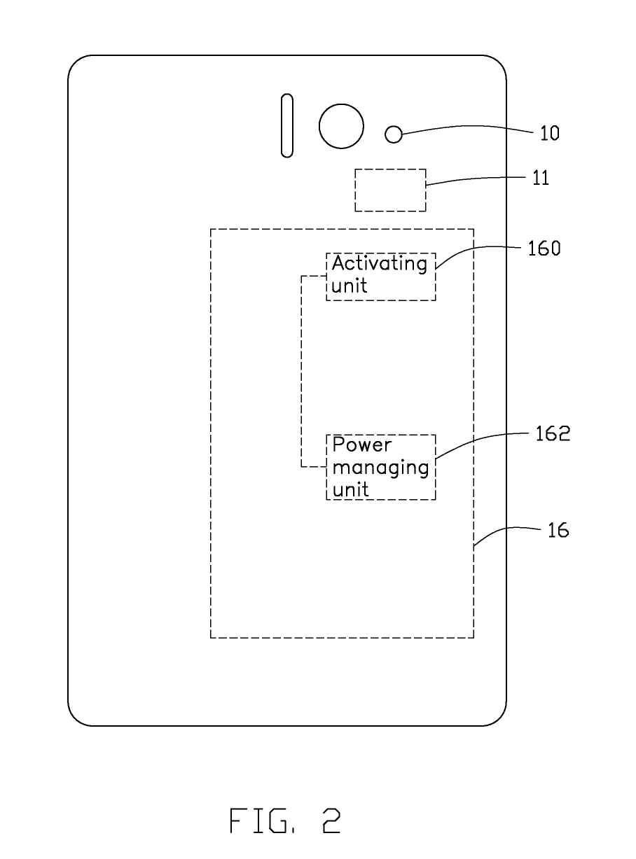

FIGS. 1 and 2 illustrate an embodiment of a device 1 for detecting air pollutants. In at least one embodiment, the device 1 can detect a quantity of carbon monoxide in the air. In other embodiments, the device 1 can detect a quantity of any other type of pollutant, such as sulfur dioxide, natural gas, methane, sulfate, or ammonium nitrate, for example. The device 1 can be a mobile phone, a tablet computer, or the like. An air passageway 10 can be defined in a main body of the device 1. Air can be fed into the device 1 through the air passageway 10. The device 1 can include an air detection unit 11, an air detection system 12, a gyroscope 13, an accelerometer 14, a display 15, a printed circuit board (PCB) 16, and a central processing unit (CPU) 17. The air detection unit 11 can detect a quantity of the air pollutant in the air fed into the device 1. The air detection system 12 can determine whether the quantity of the air pollutant is greater than a threshold value. The gyroscope 13 and the accelerometer 14 can cooperatively detect positions and moving distances of the device 1 when the quantity of the air pollutant is greater than the threshold value. The PCB 16 can include an activating unit 160 and a power managing unit 162. The activating unit 160 can turn on the gyroscope 13 and the accelerometer 14, and the power managing unit 162 can manage power of the device 1. In at least one embodiment, the air detection system 12 can calculate a relationship between positions and moving distance of the device 1 and the quantity of the air pollutant.

The air detection system 12 can include a calculating module 120 and a displaying module 122. The modules 120-122 can include one or more software programs in the form of computerized codes stored in the storage system (not shown) of the device 1. The computerized codes can include instructions executed by the CPU 17 to provide functions for the modules 120-122.

The calculating module 120 can determine whether the quantity of the air pollutant is greater than the threshold value. In detail, the threshold value of the air pollutant corresponds to a predetermined resistance between two electrodes of the air detection unit 11. In at least one embodiment, a change in the resistance between the two electrodes of the air detection unit 11 is caused by a product of a catalytic reaction between the air pollutant and a material of the air detection unit 11 when the product passes through a transistor gate electrode of the air detection unit 11. Thus, a larger quantity of the air pollutant corresponds to a higher degree of the resistance between the two electrodes. A smaller quantity of the air pollutant corresponds to a lower degree of the resistance between the two electrodes. When the quantity of the air pollutant is greater than the threshold value, the activating unit 160 of the PCB 16 can turn on the gyroscope 13 and the accelerometer 14 to detect the positions and moving distances of the device 1. In at least one embodiment, the calculating module 120 can calculate the relationship between the quantity of the air pollutant and the positions and moving distance of the device 1 relative to a horizontal orientation and a vertical orientation of the device 1.

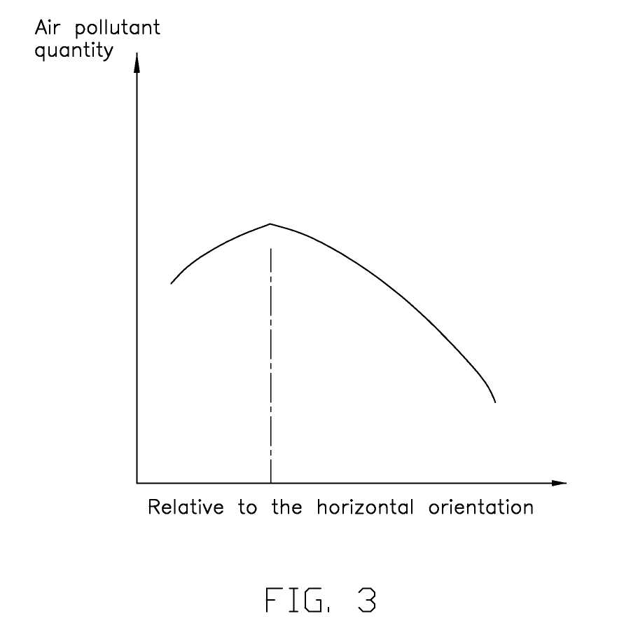

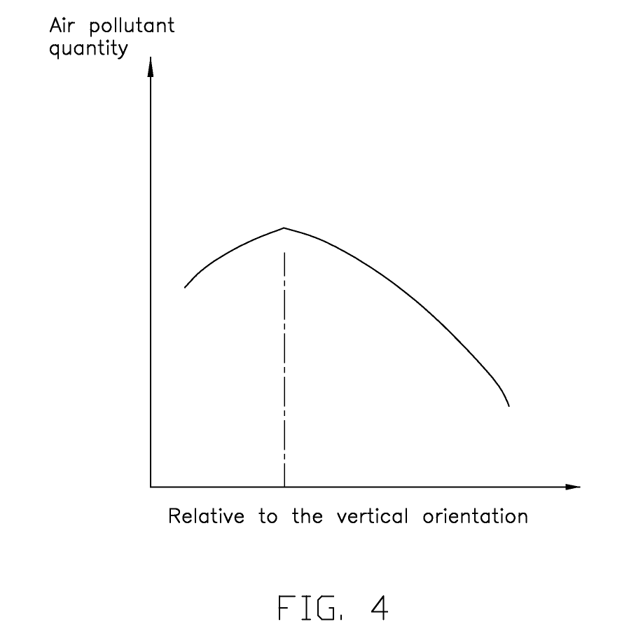

Referring to FIGS. 3 and 4, after the gyroscope 13 and the accelerometer 14 are turned on, the calculating module 120 can calculate the relationship between the quantity of the air pollutant and the positions and moving distances of the device 1. As the device 1 moves, the calculating module 120 obtains the positions of the device 1, and calculates the quantity of the air pollutant relative to the horizontal orientation and the vertical orientation of the device 1. In at least one embodiment, the calculating module 120 can calculate the quantity of the air pollutant at predetermined distance intervals of the device 1 moving. In other embodiments, the calculating module 120 can calculate the quantity of the air pollutant at predetermined time intervals of the device 1 moving.

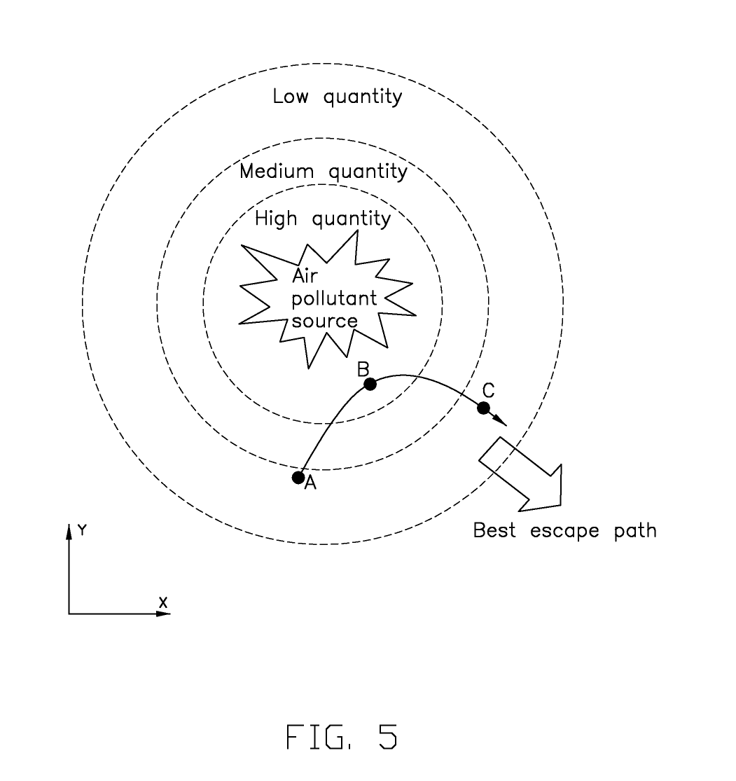

Referring to FIG. 5, the displaying module 122 can display the relationship between the quantity of the air pollutant and the positions and moving distances of the device 1 on the display 15. In at least one embodiment, the relationship is displayed as a line graph, and points of the line graph correspond to the quantity of the air pollutant relative to the vertical orientation verses the quantity of the air pollutant relative to the horizontal orientation. In the illustrated embodiment, the device 1 at a point A corresponds to a first relationship calculated by the calculating module 120 after the gyroscope 13 and the accelerometer 14 have been turned on. A point B corresponds to a second relationship calculated by the calculating module 120. As illustrated, the quantity of the air pollutant increases relative to both the vertical orientation and the horizontal orientation of the device 1 as the device 1 is moved a distance from point A to point B. A point C corresponds to a third relationship calculated by the calculating module 120. As illustrated, the quantity of the air pollutant decreases relative to both the vertical and the horizontal orientation of the device 1 as the device 1 is moved a distance from point B to point C. Thus, a user of the device 1 can know a best escape path away from an air pollutant source.

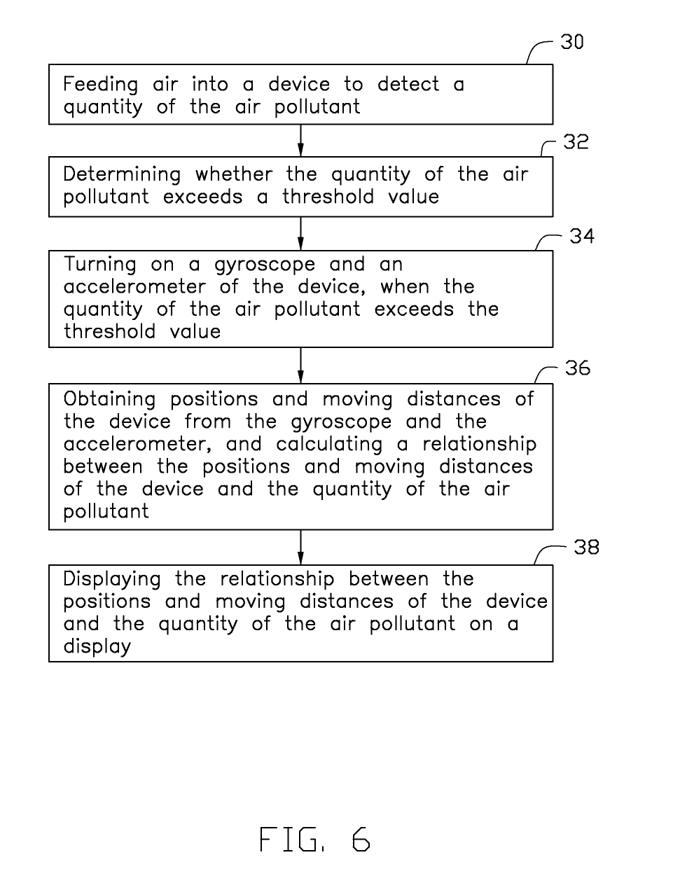

FIG. 6 illustrates a flowchart of an exemplary method for detecting an air pollutant. The example method is provided by way of example, as there are a variety of ways to carry out the method. The method described below can be carried out using the configurations illustrated in FIGS. 1-5, for example, and various elements of these figures are referenced in explaining the example method. Each block shown in FIG. 6 represents one or more processes, methods, or subroutines carried out in the example method. Furthermore, the illustrated order of blocks is by example only, and the order of the blocks can be changed. Additional blocks may be added or fewer blocks may be utilized, without departing from this disclosure. The example method can begin at block 30.

At block 30, air can be fed into a device to detect a quantity of the air pollutant. A type of the air pollutant can be predetermined.

At block 32, whether the quantity of the air pollutant exceeds a threshold value can be determined. In detail, the threshold value of the air pollutant corresponds to a predetermined resistance between two electrodes of an air detection unit of the device. In at least one embodiment, a change in the resistance between the two electrodes is caused by a product of a catalytic reaction between the air pollutant and a material of the air detection unit when the product passes through a transistor gate electrode of the air detection unit. Thus, a larger quantity of the air pollutant corresponds to a higher degree of the resistance between the two electrodes. A smaller quantity of the air pollutant corresponds to a lower degree of the resistance between the two electrodes.

At block 34, a gyroscope and an accelerometer of the device can be turned on when the quantity of the air pollutant exceeds the threshold value.

At block 36, positions and moving distances of the device can be obtained from the gyroscope and the accelerometer, and a relationship between the quantity of the air pollutant and the positions and moving distances of the device can be calculated. The relationship can be calculated relative to a horizontal orientation and a vertical orientation of the device. In at least one embodiment, the relationship can be calculated at predetermined distance intervals of the device moving. In other embodiments, the relationship can be calculated at predetermined time intervals of the device moving.

At block 38, the relationship between the quantity of the air pollutant and the positions and moving distances of the device can be displayed on a display. In at least one embodiment, the relationship is displayed as a line graph. Points of the line graph can correspond to the relationship between the quantity of the air pollutant relative to the vertical orientation of the device verses the relationship between the quantity of the air pollutant relative to the horizontal orientation of the device.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A method for detecting an air pollutant, the method comprising:
feeding air into a device to detect a quantity of the air pollutant;
determining whether the quantity of the air pollutant exceeds a threshold value;
activating a gyroscope and an accelerometer of the device when the quantity of the air pollutant exceeds the threshold value;
obtaining, from the gyroscope and the accelerometer, a position and a moving distance of the device, and calculating a relationship between the position and moving distance of the device and the quantity of the air pollutant; and
displaying the relationship between the position and moving distance of the device and the quantity of the air pollutant.

2. The method as in claim 1, wherein a type of the air pollutant is predetermined, and the type of the air pollutant comprises at least one of nitrogen dioxide, sulfur dioxide, carbon monoxide, natural gas, methane, sulfate, and ammonium nitrate.

3. The method as in claim 1, wherein:
the threshold value of the air pollutant corresponds to a predetermined resistance between two electrodes of an air detection unit of the device; and
a change in the resistance between the two electrodes is caused by a product of a catalytic reaction between the air pollutant and a material of the air detection unit passing through a transistor gate electrode of the air detection unit.

4. The method as in claim 3, wherein:
a change of the quantity of the air pollutant corresponds to the change in the resistance between the two electrodes of the air detection unit; and
a higher degree of the resistance between the two electrodes of the air detection unit corresponds to a larger quantity of the air pollutant, and a lower degree of the resistance corresponds to a smaller quantity of the air pollutant.

5. The method as in claim 1, wherein the positions and moving distances of the device are detected relative to a horizontal orientation and a vertical orientation of the device.

6. The method as in claim 5, wherein the quantity of the air pollutant is calculated at predetermined distance intervals of the device moving.

7. The method as in claim 5, wherein the quantity of the air pollutant is calculated at predetermined time intervals of the device moving.

8. The method as in claim 5, wherein the relationship between the quantity of the air pollutant and the position and moving distance is displayed as a line graph on a display.

9. The method as in claim 8, wherein:
points of the line graph correspond to a quantity of the air pollutant relative to the vertical orientation verses a quantity of the air pollutant relative to the horizontal orientation.

10. A device for detecting air pollutants, the device comprising:
a gyroscope;
an accelerometer;
a display;
a printed circuit board;
a central processing unit;
a main body defining an air passageway, the air passageway configured to feed air into the device;
an air detection unit configured to detect a quantity of an air pollutant from the air fed into the device; and
an air detection system configured to:
determine whether the quantity of the air pollutant exceeds a threshold level;
activate the gyroscope and the accelerometer when the quantity of the air pollutant exceeds the threshold level; and
calculate a relationship between a position and a moving distance of the device and the quantity of the air pollutant;
wherein the position and the moving distance of the device are detected by the gyroscope and the accelerometer;
wherein the air detection system is executed by the central processing unit;
wherein the air detection system displays the relationship between the position and moving direction of the device and the quantity of the air pollutant on the display; and
wherein a type of the air pollutant is predetermined.

11. The device as in claim 10, wherein the air detection system comprises:
a calculating module configured to calculate the quantity of the air pollutant from the air detection unit, and calculate the relationship between the positions and moving distances of the device and the quantity of the air pollutant; and
a displaying module configured to display the relationship between the positions and moving directions of the device and the quantity of the air pollutant on the display.

12. The device as in claim 11, wherein:
the threshold value of the air pollutant corresponds to a predetermined resistance between two electrodes of an air detection unit of the device;
a change of the resistance between the two electrodes is caused by a product of a catalytic reaction between the air pollutant and a material of the air detection unit passing through a transistor gate electrode of the air detection unit;
a change of quantity of the air pollutant corresponds to the change of the resistance between the two electrodes of the air detection unit; and
a higher degree of the resistance between the two electrodes of the air detection unit corresponds to a larger quantity of the air pollutant, and a lower degree of the resistance corresponds to a smaller quantity of the air pollutant.

13. The device as in claim 10, wherein the printed circuit board comprises:
an activating unit configured to turn on the gyroscope and the accelerometer when the quantity of the air pollutant is greater than the threshold value; and
a power managing unit configured to manage power of the device.

14. The device as in claim 13, wherein the air passageway, the air detection unit, the gyroscope, the accelerometer, the activating unit, and the display are electrically coupled together through a data bus of the device.

15. The device as in claim 10, wherein the positions and moving distances of the device are detected relative to a horizontal orientation and a vertical orientation of the device.

16. The device as in claim 15, wherein the quantity of the air pollutant is calculated at predetermined distance intervals of the device moving.

17. The device as in claim 15, wherein the quantity of the air pollutant is calculated at predetermined time intervals of the device moving.

18. The device as in claim 15, wherein the relationship between the quantity of the air pollutant and the positions and moving distances of the device is displayed as a line graph on the display.

19. The device as in claim 18, wherein:
points of the line graph correspond to a quantity of the air pollutant relative to the vertical orientation verses a quantity of the air pollutant relative to the horizontal orientation.

20. The device as in claim 10, wherein the type of the air pollutant comprises at least one of nitrogen dioxide, sulfur dioxide, carbon monoxide, natural gas, methane, sulfate, and ammonium nitrate.
